(19) United States
(12) Patent Application Publication
(10) Pub. No.: US 2008/0139918 A1
(43) Pub. Date: Jun. 12, 2008

(54) Device and method for producing images

(76) Inventor: Andreas Berting, Radmuhl (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/999,650

(22) Filed: Dec. 6, 2007

(30) Foreign Application Priority Data
Dec. 8, 2006 (DE) 10 2006 057 987.9

(51) Int. Cl. A61B 5/02; A61B 5/053

(52) U.S. Cl. 600/407; 600/547

(57) ABSTRACT

The invention relates to an image-producing apparatus comprising an imaging apparatus for the production of images on a patient's body to be examined, an impedance cardiograph for the determination of at least one specific period of cardiac activity, a connection line between the impedance cardiograph and the imaging apparatus for the transmission of information on the at least one specific period and a control means in or on the imaging apparatus, which is intended to control the image acquisition by the imaging apparatus during the specific period. The invention also includes corresponding methods and uses.

### CROSS REFERENCE TO RELATED APPLICATIONS

This application claims priority of German application No. 10 2006 057 987.9 filed Dec. 8, 2006, which is incorporated by reference herein in its entirety.

### FIELD OF THE INVENTION

The present invention is directed at an image-producing apparatus with an impedance cardiograph and a method for producing images using an impedance cardiogram.

### BACKGROUND OF THE INVENTION

With various image-producing methods for examining the bodies of, for example, economically useful animals or human patients, it is necessary or at least advantageous to synchronize the image acquisition to be performed by the particular imaging apparatus used with the cardiac activity of the patient to be examined in order to improve the quality of the image acquisition and data records calculated therefrom for imaging the patient's body. The most typical examples of this kind of necessary or desirable synchronization should be considered in particular to be image-producing methods which are themselves used for the production of cardiac images, such as, for example, angiography systems or computer tomographs. The strong movement of the heart during contraction does not permit clean image production with methods requiring lengthy exposure times or irradiation times, so that image acquisition is preferably performed during a diastolic phase of the heart, that is in a state of relaxation. In the prior art, the R wave of an electrocardiogram is usually used for the synchronization of the image-acquiring method with the cardiac activity. The diastolic cardiac phase is usually required for the depiction of the cavities of the heart and in particular the coronary arteries.

However, it is obvious that corresponding methods do not have to be restricted to examinations of the actual heart, but can also relate to the production of images of other parts of the body insofar as their acquisition can be impaired by the cardiac activity.

However, up to now, there has been a problem, particularly in the case of patients with an unstable cardiac rhythm, in that it has been impossible, or only restrictedly possible, to predict the next R wave in the ECG, so that up to now the acquisition time of the image data either does not fully utilize the diastolic phase (for safety reasons, the acquisition time is reduced in order not to extend beyond the diastolic phase) or extends into the in next systole (which impairs the image quality).

### SUMMARY OF THE INVENTION

The object of the invention is to provide a device and a method for image acquisition on a patient's body, which could be influenced by cardiac activity, which is able to identify and take account of more reliably determined cardiac phases with correct timing.

According to the invention, this object is achieved by the independent claims. Further advantageous embodiments, aspects and details of the present invention may be found in the dependent claims, the description and the attached drawings.

The invention is based on the principle of using an impedance cardiogram (ICG) and an impedance cardiograph, produced thereby, to control the image production instead of an electrocardiogram (ECG).

It is precisely the synchronization with the diastolic cardiac phase that can be performed in a better way with the impedance cardiogram than it can with an ECG.

Correspondingly, in a first aspect, the invention is directed at an image-producing apparatus comprising: an imaging apparatus for the production of images on a patient's body to be examined, an impedance cardiograph for determining at least one specific period of the cardiac activity, a connection line between the impedance cardiograph and the imaging apparatus for the transmission of information on the at least one specific period and a control means in or on the imaging apparatus which is intended to control an image acquisition by the imaging apparatus during the specific period.

For the purpose of the invention, an imaging apparatus should be understood as being any apparatus which is able to visualize two- or three-dimensional images or sequences in a body. A patient to be examined includes all types of patients suitable for an image-producing method, for example humans, economically useful animals, such as horses or cattle, or zoo animals, such as antelopes, etc.

For the purposes of the invention, an impedance cardiograph is a device known in principle from the prior art which utilizes the impedance of the body to pick up cardiac activities and differs in the impedance characteristics it records from the voltage characteristics of an ECG.

The connection line between the impedance cardiograph and the imaging apparatus, which is able to transmit information on the at least one specific period, can be any kind of connection between the devices, including electrical and optical lines, plus bus systems with the integration of all components in a common device housing, with centralized or decentralized control of the components.

The control means, which can be arranged in or on the imaging apparatus, can be an additionally provided control means in combination with a conventional imaging apparatus, or an integrated module in an imaging apparatus or, by means of specific programming, the generally used control device in an imaging apparatus (for example a PC for controlling the imaging apparatus).

Obviously, the different components of the image-producing apparatus can be combined in the same device and share a plurality of components, for example the control apparatuses required to operate the individual subapparatuses which can also be used for the synchronization of the two main components ‘imaging apparatus’ and ‘impedance cardiograph’ and also other components such as, for example, power supply, etc.

In a preferred embodiment, the imaging apparatus is intended to produce images of the heart, but it is also possible to use it for other parts of the body, in particular in cases when the production of the images would otherwise be impaired by the cardiac activity. In particular, it is preferred that the imaging apparatus is an angiograph or a tomograph, such as an X-ray computer tomograph.

Generally, features of the impedance cardiogram can be used to determine the starting time and/or the finishing time of the specific period.

There are also numerous options for the specific use of the information contained in an impedance cardiogram. For example, in a preferred embodiment, a start of the specific period can lie within the region of the O wave of the impedance cardiogram. It is also possible for a start of the specific period to lie within the region of the X point of the impedance cardiogram.

Preferably, one end of the specific period lies within the region of the A wave of the impedance cardiogram. In combination with the above preferred embodiment, therefore, one possible region between the O wave and A wave and another between the X point and the A wave are occupied, it being obvious that other periods can also be used as specific periods.

Furthermore, one end of the specific period can be averaged on the basis of preceding impedance cardiogram cycles for a specific impedance cardiogram cycle, in which the image acquisition can take place.

It is also possible for one end of the specific period to be determined on the basis of pre-specified periods after the start of the specific period, that is by the preliminary definition of the length of the specific period for which then only the start is also determined, wherein the end is obtained from pre-specified periods.

In particular, it is preferred that the specific period is the diastole of the heart. However, obviously it is also possible to use other periods depending on the objective of the examination, the imaging apparatus used or further questions and the invention should not be restricted to image acquisition during diastolic periods.

In addition to an ICG, in a further preferred embodiment of the invention, an ECG can be used in order further to improve the precision of the determination of the cardiac activity, whereby a combination of the information from the two cardiograms increases the number of possible criteria when generating the information on the cardiac activity.

In a further aspect, the invention is directed at the use of an impedance cardiograph for controlling the production of images of an imaging apparatus for producing images, wherein the impedance cardiogram acquired by the impedance cardiograph is used to define a specific period which is intended to control an image acquisition by the imaging apparatus.

The invention is also directed at a use of at least the O wave of the impedance cardiogram of a patient to be examined to determine the start of a cardiac phase suitable for producing images with a tomograph. Everything described above with respect to the apparatus according to the invention also applies to the uses according to the invention and vice versa, so that alternate reference is made.

Finally, in one aspect, the invention is directed at a method to produce images comprising the steps:
acquisition of an impedance cardiogram of a patient to be examined;
determination of at least one specific period of cardiac activity by means of the impedance cardiogram; and
control of an image acquisition by an imaging apparatus for the production of images on the patient's body during the specific period

Everything described above with respect to the apparatus according to the invention also applies to the method according to the invention and vice versa, so that alternate reference is made.

Features of the impedance cardiogram are used to determine the starting time and/or finishing time of the specific period.

Preferably, one start of the specific period is placed in the region of the O wave of the impedance cardiogram, which generally marks the start of a diastolic cardiac phase. The region of the X point of the impedance cardiogram, which coincides with the time of the aortic valve closure, can also be used to define the start of the specific period.

One end of the specific period can be placed in the region of the A wave of the impedance cardiogram in order to achieve the termination of the image acquisition at the correct time before the onset of the systole. It is particularly preferable for the specific period to be the diastole of the heart.

### BRIEF DESCRIPTION OF THE DRAWINGS

The following describes the invention using specific exemplary embodiments wherein reference is made to the attached drawings, which show:

FIG. 1 the structure of an image-producing apparatus according to the invention

FIG. 2 shows the application of electrodes for an impedance cardiograph on a human patient's body and

FIG. 3 shows the ECG and ICG in temporal synchronicity to explain the simpler diastole determination.

### DETAILED DESCRIPTION OF THE INVENTION

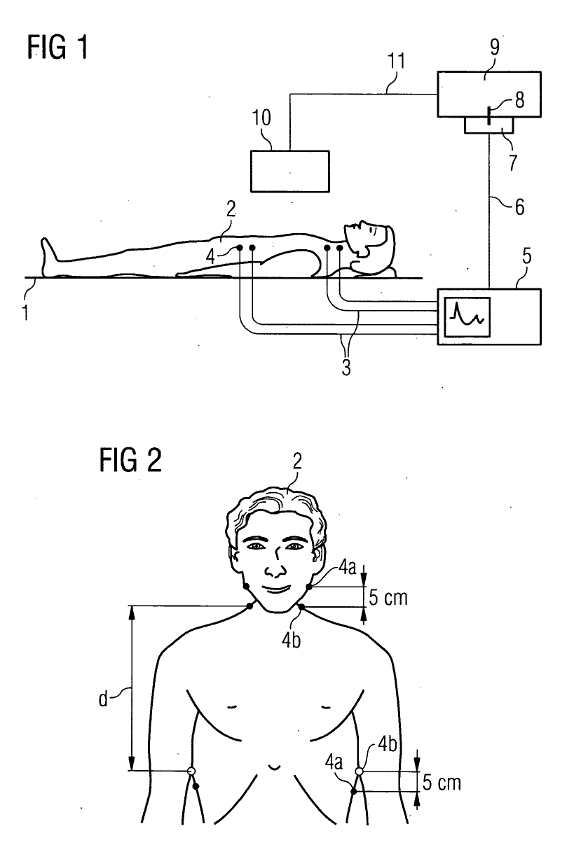

FIG. 1 shows the basic principle of the present invention. Shown lying on a support 1, there is a patient 2 who is connected via lines 3 and body electrodes 4 to an impedance cardiograph 5. The impedance cardiograph 5 is connected via a connection line 6 to a control device 7 which is connected via a further connection 8 to the control device 9 of an imaging apparatus and which, in addition to the actual control apparatus 9 also comprises a measuring device 10 and a connection line 11 between the control apparatus 9 and measuring device 10. The connection line 6 transmits the impedance cardiogram signals derived from the patient to the control apparatus which uses conventional pattern recognition mechanisms to determine the characteristic elements of the cardiogram in their temporal sequence and from these in turn calculates from preselected secondary conditions, defining, for example, a physician, the desired specific period that appears suitable for an examination and forwards the corresponding activation or deactivation signals to the control apparatus 9 of the imaging apparatus.

FIG. 2 is a front view of a human patient to be examined, including the electrode arrangement for the acquisition of the impedance cardiogram. The arrangement comprises current electrodes 4a and measuring electrodes 4b arranged on the neck with a spacing of 5 cm plus further current electrodes 4a and measuring electrodes 4b arranged on the side of the torso, once again with a spacing of 5 cm. This results in the electrode spacing represented by the double-headed arrow. This arrangement is known in principle and is only shown here for purposes of completeness. Typically, an alternative current of 1 to 4 mA with a frequency of 50 to 100 kHz (upper beta dispersion outflow region) flows through the current electrodes.

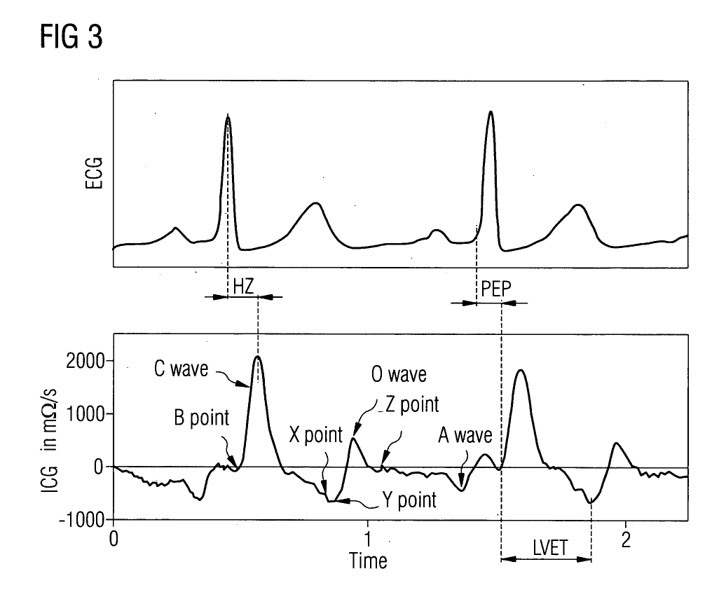

FIG. 3 shows, in the upper half, the temporal course of a conventional ECG and, in the lower half, an ICG which can be used according to the invention. The ICG represents impedance changes in the thorax which are caused by the haemodynamics.

Compared to a phono-cardiogram, the ICG displays distinctive curve points even in the diastolic phase, as shown in

FIG. 3. For example, the X point coincides with the time of the aortic valve closure, the 0 wave maximum corresponds to the mitral valve opening (MV) and the A wave minimum is temporally congruent with the atrial systole. Further distinctive points are the B point as the first heart tone, the C wave as a systolic wave, the Y point as the end of the second heart tone and the Z point as the third heart tone. Therefore, due to the numerous features in the cardiogram, the ICG enables the diastolic cardiac phase to be utilized efficiently and to a large extent in its full length without unreliable predictions.

The general use of the ICG for synchronization in arrhythmic patients requires an atrial contraction before the ventricular systole, which is present, for example, in the case of sinus arrhythmia or supraventricular extrasystoles. In the case of an absolute arrhythmia, with which atrial fibrillations are also associated, the exact determination of the aortic valve closure or of the early-diastolic inflow should also be mentioned as a further advantage of the invention. The same applies to ventricular extrasystoles. In this case, for example, the R wave in the conventional ECG would signify the end of the image acquisition (combined use of ECG and ICG for synchronization).

The advantages of the present invention consist in the full utilization of the diastolic cardiac phase for the image acquisition and quite generally in the possibility of using a larger number of features for determining the current condition of the heart in the cycle. In particular, therefore, it makes it possible to make efficient use of the diastolic phase in arrhythmic patients as well, which represents a great advance for diagnosis and interventional therapy. Therefore, in particular in the case of supra-ventricular extrasystoles, more efficient utilization of the diastolic cardiac phase for image acquisition may be expected.

1.-25. (canceled)

26. An image-producing apparatus, comprising:
an imaging apparatus that acquires an image on a body of a patient;
an impedance cardiograph that determines a specific period of a cardiac activity of the patient;
a connection line between the impedance cardiograph and the imaging apparatus that transmits information; and
a control device that controls the acquisition of the image during the specific period.

27. The image-producing apparatus as claimed in claim 26, wherein the body of the patient is a heart of the patient.

28. The image-producing apparatus as claimed in claim 26, wherein the imaging apparatus is an angiograph or a tomography.

29. The image-producing apparatus as claimed in claim 28, wherein the tomograph is an X-ray computer tomograph.

30. The image-producing apparatus as claimed in claim 26, wherein an impedance cardiogram of the patient is recorded by the impedance cardiograph for determining a starting time or a finishing time of the specific period.

31. The image-producing apparatus as claimed in claim 30, wherein the starting time is within a region of a X point of the impedance cardiogram or a region of an O wave of the impedance cardiogram.

32. The image-producing apparatus as claimed in claim 30, wherein the finishing time is within a region of an A wave of the impedance cardiogram, or is an average of preceding impedance cardiogram cycles, or is determined by a pre-specified period after the specific period starts.

33. The image-producing apparatus as claimed in claim 26, wherein the specific period is a diastole of a heart of the patient.

34. The image-producing apparatus as claimed in claim 26, wherein the control device is arranged in or on the imaging apparatus.

35. A method for acquiring an image of a body of a patient, comprising:
recording an impedance cardiogram of the patient;
determining a specific period of a cardiac activity of the patient based on the impedance cardiogram;
acquiring the image of the body of the patient by an imaging apparatus; and
controlling the acquisition of the during the specific period.

36. The method as claimed in claim 35, wherein the body of the patient is a heart of the patient.

37. The method as claimed in claim 35, wherein the imaging apparatus is an angiograph or a tomography.

38. The method as claimed in claim 35, wherein a starting time or a finishing time of the specific period is determined by the impedance cardiogram.

39. The method as claimed in claim 38, wherein the starting time is within a region of a X point of the impedance cardiogram or a region of an O wave of the impedance cardiogram.

40. The method as claimed in claim 38, wherein the finishing time is within a region of an A wave of the impedance cardiogram, or is an average of preceding impedance cardiogram cycles, or is determined by a pre-specified period after the specific period starts.

41. The method as claimed in claim 35, wherein the specific period is a diastole of a heart of the patient.
